(19) United States
(12) Patent Application Publication
(10) Pub. No.: US 2025/0361627 A1
(43) Pub. Date: Nov. 27, 2025

(54) Electrochemical Alcohol Nitration Systems and Methods

(71) Applicants: Yale University, New Haven, CT (US); Nalas Engineering Services, Inc., Centerbrook, CT (US)

(72) Inventors: Fang Lin, New Haven, CT (US); Paul Anastas, New Haven, CT (US); Matthew Burk, New Haven, CT (US); Mark Plummer, Westbrook, CT (US); Josiah Smith, Lyme, CT (US); Eric Weidman, San Jose, CA (US); Omar Radwan, New Haven, CT (US)

(73) Assignees: Yale University, New Haven, CT (US); Nalas Engineering Services, Inc., Centerbrook, CT (US)

(21) Appl. No.: 19/135,314

(22) PCT Filed: Nov. 27, 2023
(86) PCT No.: PCT/US2023/081103

§ 371 Date: Jun. 3, 2025

Related U.S. Application Data
(60) Provisional application No. 63/429,016, filed on Nov. 30, 2022.

(51) Int. Cl. C25B 3/09; C07C 203/04; C07C 203/06; C25B 3/03; C25B 3/07; C25B 9/15; C25B 9/19; C25B 15/08

(52) U.S. Cl. CPC C25B 3/09; C07C 203/04; C07C 203/06; C25B 3/03; C25B 3/07; C25B 9/15; C25B 9/19; C25B 15/08

(57) ABSTRACT

Electrochemistry is used to generate active nitrating species from nitrate salt in situ in an aprotic solvent to eliminate acidic and/or toxic waste streams associated with the production of energetic materials. The systems/methods perform alcohol nitration without using nitric acid and/or sulfuric acid. As a result, the systems/methods may be operated under milder conditions (e.g., room temperature and ambient pressure). In addition, the disclosed systems/methods offer high product selectivity via controlling electrolysis potential. The electrochemical synthetic method is scalable, highly amenable to continuous processing and can make use of inexpensive feedstocks, making the systems/methods well-suited to large-scale manufacture.

### 2. CROSS-REFERENCE TO RELATED APPLICATION

The present application claims priority benefit to a U.S. provisional patent application entitled “Electrochemical Alcohol Nitration Systems and Methods,” filed on Nov. 30, 2022, and assigned Ser. No. 63/429,016. The entire content of the foregoing U.S. provisional patent application is incorporated herein by reference.

### 1. GOVERNMENT SUPPORT

This invention was made with government support under SERDP SEMS WP20-C2-1010 awarded by the Strategic Environmental Research and Development Program within the U.S. Department of Defense. The government has certain rights in the invention.

### BACKGROUND

3. Technical Field

The present disclosure is directed to systems and methods for electrochemical nitration to produce energetic materials. The disclosed methods are “greener” than conventional methods, eliminating the use of large excesses of nitric and sulfuric acids (commonly referred to as mixed acid nitration) associated with conventional nitration chemistry methods. The use of active nitrating agents, e.g., nitrating agents electrolytically generated from nitrogen tetroxide (N₂O₄), or a nitrate salt in an aprotic solvent, has the potential to provide improved selectivity while reducing or eliminating acidic and/or toxic waste streams. The disclosed systems/methods may have broad application, e.g., extending to the nitration of a variety of substrates, including aromatic moieties and polyols, maximizing impact across a range of energetic materials, e.g., the portfolio of energetic materials currently in use by the United States Department of Defense (US DoD).

### 4. Background Art

Nitrated organic compounds comprise a significant portion of energetic materials used in certain industries and government agencies, e.g., US DoD. Despite their critical importance, little progress has been made toward reducing the negative environmental impact of their manufacture. Chemical nitration processes used to produce these compounds have remained largely unchanged for decades, typically relying on mixed acid nitration chemistry where nitric acid in combination with other acidic media (e.g., sulfuric acid) is used as the nitrating agent in significant stoichiometric excess. A result of these processes is the generation of over 10 million pounds of spent acid each year at Army ammunition plants that require additional processing before disposal. Beyond the large quantity of spent acid waste, the use of nitric acid as a nitrating reagent can create issues of chemical selectivity. For example, the nitration of toluene to TNT when using mixed acid nitration provides undesirable amounts of meta nitro species (e.g., meta nitrotoluene species) as well as generating significant waste streams. These meta nitro species are generally removed through a sulfiting process, which selectively destroys these meta isomers, but in the process creates additional waste called “red water” that is both hazardous and expensive to treat.

A need exists for improved/alternative systems and methods for the production of energetic materials, including systems/methods that substantially reduce or eliminate acidic and/or toxic waste streams and improve selectivity in synthesis. These and other objectives are met according to the present disclosure.

### SUMMARY

The present disclosure is directed to systems and methods for electrochemical nitration, e.g., for the production of energetic materials. The disclosed methods are “greener” than conventional methods, eliminating the use of large excesses of nitric and sulfuric acids associated with conventional nitration chemistry methods, including potential elimination of the use of sulfuric acid entirely.

According to the present disclosure, electrochemistry is used to generate active nitrating species from N₂O₄ or a nitrate salt in situ in an aprotic solvent. The disclosed methods/systems eliminate or substantially reduce acidic and/or toxic waste streams associated with the production of energetic materials. As disclosed herein, electrochemistry can be used to generate active nitrating species from a nitrite salt alone, e.g., when the salt is silver nitrite (AgNO₂).

In an exemplary embodiment, AgNO₂ systems/methods may be used to perform alcohol nitration to form nitrate esters and anisole nitration to form nitroanisole (NA) directly in the anode compartment of an electrochemical apparatus. Alternatively, for example, electrolysis of N₂O₄ and a lithium nitrate salt system in aprotic solvent(s), may be used to form N₂O₅ (nitronium nitrate dinitrogen pentoxide). The disclosed electrolysis may be used, for example, in nitration of alcohols to nitrate ester, nitration of anisole to nitroanisole (NA) or dinitroanisole (DNAN), and nitration of toluene to mononitrotoluene (MNT) or dintrotoluene (DNT) without the need for mixed acid nitration methods.

In alternative embodiments, electrolysis of N₂O₄ with other charge carrying salts besides lithium nitrate may be undertaken to produce other nitrating agents. For example, lithium triflimide as a charge carrying salt yields nitronium triflimide, and lithium triflate as a charge carrying salt yields nitronium triflate.

The disclosed systems/methods may be operated under mild conditions (e.g., low temperature (−15° C.-25° C.) and ambient pressure). In addition, the disclosed systems/methods offer high product selectivity that can be effectuated, for example, by controlling electrolysis potential. The disclosed electrochemical synthetic method is scalable, highly amenable to continuous processing and can make use of inexpensive feedstocks. making the systems/methods well-suited to large-scale manufacture.

The disclosed systems/methods have broad application, e.g., allowing production of nitrate esters that serve as important components for military and commercial energetic materials and extending to the nitration of a variety of substrates, including aromatic hydrocarbons and polyols, maximizing impact across a range of energetic materials and other commercial needs, e.g., medicines. Indeed, nitrate esters have been shown to have vasodilatory effects in humans and have been used for treatments of ailments such as angina.

Additional features, functions and benefits of the disclosed systems and methods will be apparent from the description which follows.

### BRIEF DESCRIPTION OF THE FIGURES

To assist those of skill in the art in making and using the systems and methods of the present disclosure, reference is made to the appended figures wherein:

FIG. 1 shows a divided H-cell with a reference electrode, a Pt foil anode with a glass divider, and a coiled Pt wire cathode for use according to the present disclosure;

FIG. 2 shows an advance flow reactor that may be used for nitration of aromatic and alcohol substrates according to the present disclosure;

FIG. 3 shows a flow electrolysis system that includes two reactors and a micro flow cell for use according to the present disclosure;

FIG. 4 sets forth NMR resonances of 1,2,3-nitroglycerin spectrum in an experimental synthesis according to the present disclosure;

FIG. 5 sets forth NMR spectrum showing relevant down field signals of BTTN formed from N₂O₅ produced at the anode of a two-piece membrane divided H-cell according to the present disclosure;

FIG. 6 is a schematic depiction of an undivided electrolysis cell set up according to the present disclosure; and

FIG. 7 is a schematic depiction of a divided H cell where the working electrode is the anode, and the counter electrode is the cathode.

### DETAILED DESCRIPTION

According to exemplary embodiments of the present disclosure, electrochemistry-based systems and methods form active nitrating species, e.g., N₂O₅ or other nitronium species, through a synthesis that includes an aprotic solvent that expends only electrons in the presence of a charge carrying lithium salt and readily available N₂O₄ or a nitrate salt. The aprotic solvents used according to the disclosed systems and methods, e.g., acetonitrile (ACN) with varying amounts of ethylene carbonate (EC) or other alkyl carbonate, or other co-solvents, and the charge carrying lithium salt used according to the disclosed systems and methods, are all recoverable.

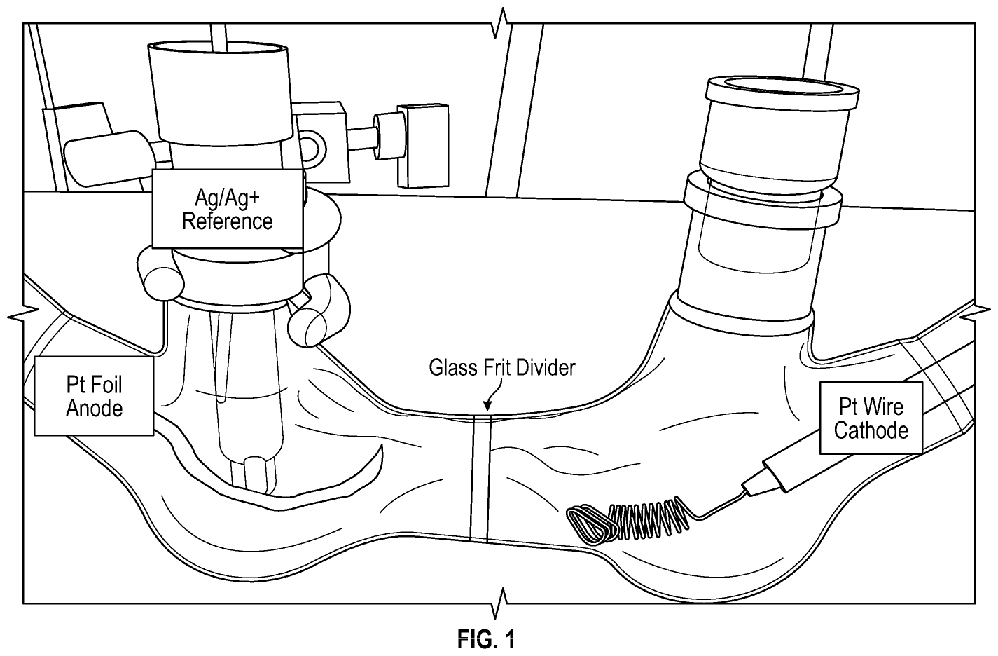

In exemplary implementations, the disclosed electrochemical system/method may employ a divided H-cell with a reference electrode and Pt foil anode with a glass or membrane divider, a coiled Pt wire cathode and Ag/Ag+ reference electrode, e.g., as shown in FIG. 1. In a divided H-cell of the type shown in FIG. 1, reagents may be charged into their appropriate compartments and electrolysis under constant current may be performed, which generates a nitrating species in the anode compartment. When electrolysis is nearing completion, the anolyte may be removed and added to a second reactor containing the nitration substrate, thereby forming the desired end product in a two-step process. Alternatively, electrolysis can be performed in a single pass flow electrolysis system or in a recirculating flow electrolysis system.

In exemplary implementations, reagents are charged into their appropriate compartments and electrolysis, preferably under constant current, is performed, which generates the nitrating species in the anode compartment. In a two-step process, as noted above, when electrolysis is nearing completion, the anolyte may be removed and added to a second reactor containing the nitration substrate, thereby forming the product in a two-step process. In embodiments, flow electrolysis may be used to form the nitrating species (e.g., through a single pass or recirculating system) and, when the nitrating species is formed, it may be flowed into a reactor plate, where the nitrating species is introduced to the substrate-producing products under flow conditions.

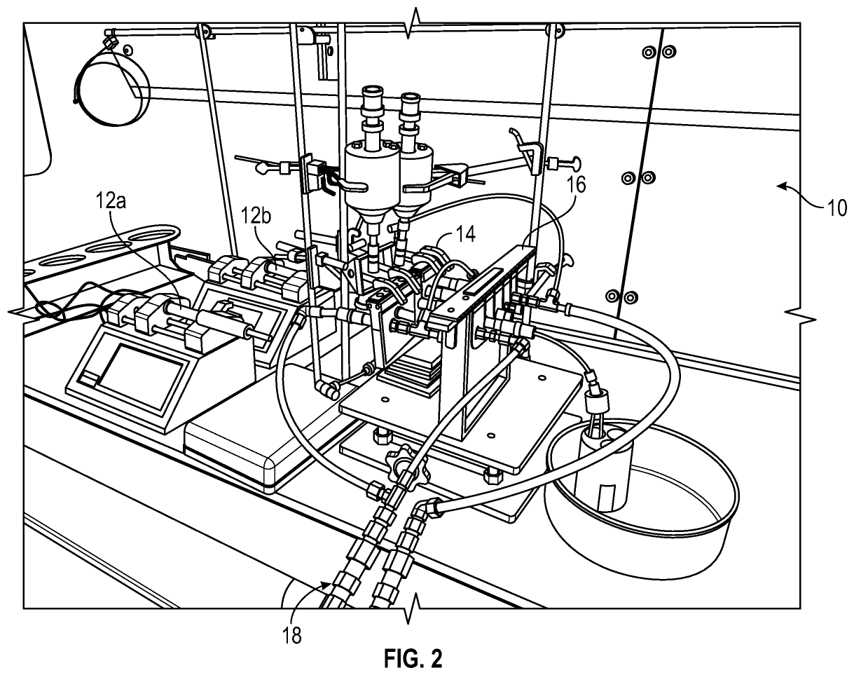

The reactor plate may take the form of an advanced flow reactor (AFR). For example, AFR system may take the form of a Corning Advanced-Flow reactor (Corning Incorporated, Corning, NY). As shown in FIG. 2, an exemplary AFR system 10 includes one or more pumps (e.g., syringe pumps) for delivering feedstock, e.g., an active nitrating species, e.g., N₂O₅ or other nitronium species, and substrate, e.g., an aromatic and/or alcohol substrate, to a precooling plate 14. The exemplary embodiment shown in FIG. 2 includes two syringe pumps 12a, 12b that operate to independently feed feedstock to AFR precooling plate 14. The AFR precooling plate 14 is in fluid communication with AFR mixing plate 16. A feed and return system 18 is provided to deliver coolant to and from the AFR precooling plate 14 and the AFR mixing plate 16. Outfeed 20 is delivered from the AFR mixing plate 16.

AFR system 10 may be used, for example, for nitration of aromatic and alcohol substrates in flow employing anolyte as a feedstock from an electrolysis system. The electrolysis system and the AFR system may be integrated for continuous operation or the two step process may be undertaken in a batch or semi-batch manner. Advantages associated with reaction in flow for nitration reactions as disclosed herein derive, at least in part, from the fact that nitration reactions are exothermic and, in flow, the exotherm is controllable as only a small amount is mixing at a given time with a high cooling surface area. Enhanced control of the heat transfer environment in flow reactor systems is advantageous as compared to batch-based nitration reactions. The enhanced control of the heat transfer environment generally translates to safer nitration reactions as well as potentially increased product selectivity due to better temperature control.

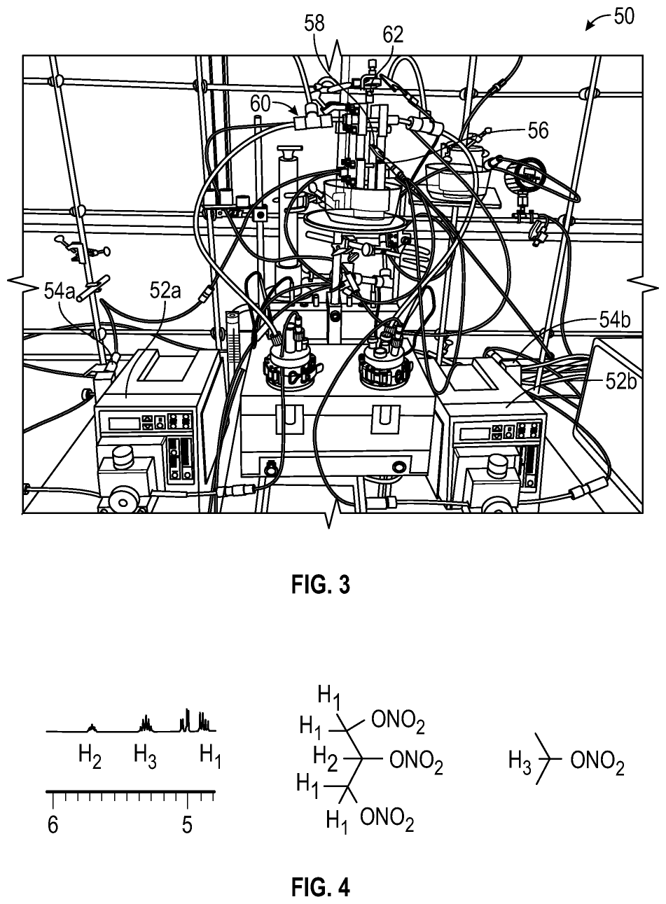

FIG. 3 shows an exemplary flow electrolysis system 50 that that includes two 100 mL EasyMax reactors (Mettler-Toledo, LLC, Columbus, OH) and an ElectroCell Micro Flow Cell for use in electrolysis reactions to produce active nitrating species. Flow electrolysis system may be used in combination with an AFR system 50, as shown in FIG. 2, but AFR system 10 and electrolysis system 50 may be used in combination with other unit operations supporting requisite reaction systems, as will be apparent to persons skilled in the art. Flow electrolysis system 50 includes pumps, e.g., peristaltic pumps 52a. 52b, that operate in conjunction with flow meters 54a, 54b, to deliver reagants to the anode 56 and cathode 58 associated with the reactor 60. A reference electrode 62 is provided in communication with the reactor 60. The reagents may be charged into their appropriate compartments and electrolysis under constant current may be performed, thereby generating a nitrating species in the anode compartment. When electrolysis is nearing completion, the anolyte may be removed and added to a second reactor as described above.

Although exemplary implementations of the disclosed systems and methods involve electrolysis carried out at or below room temperature and ambient pressure, alternative temperature and/or pressure conditions may be utilized for production of the disclosed energetic materials. A range of organic solvent soluble electrolytes may be employed according to the present disclosure including, but not limited to, tetrabutyl ammonium hexafluorophosphate (TBAPF₆), tetrabutyl ammonium tetrafluroborate (TBABF₄), and other ammonium cations. A range of lithium salts may be employed including. but not limited to. lithium nitrate, lithium triflate, lithium triflimide. The noted reactants will generally yield different nitronium species with different reactivity.

Similarly, various aprotic organic solvents and solvent mixtures may be employed, e.g., acetonitrile, ethylene carbonate, propylene carbonate, dimethoxyethane, dimethyl sulfone, sulfolane and combinations thereof. Still further, various electrode materials may be employed, e.g., carbon-based materials such as glassy carbon, carbon nanofiber paper, and graphite, and/or metals, such as copper, gold, nickel, platinum, niobium, tantalum, iridium and iridium oxide, stainless steel and the like. To further illustrate the disclosed systems and methods, reference is made to the following exemplary implementations.

The conversion of alcohols and polyols like glycerin to nitrate esters using nitronium salts has been reported. [See, Olah et al., “Synthetic Methods and Reactions; 48: Convenient and Safe Preparation of Alkyl Nitrates (Polynitrates) via Transfer Nitration of Alcohols (Polyols) with N-Nitrocollidinium Tetrafluoroborate.” In Across Conventional Lines: Selected Papers of George A Olah Volume 2, pp. 993-994. 2003. (“Olah Publication”)] However, nitronium hexafluorophosphate, even at cold temperatures, is too reactive to give nitrate esters in high yield. Moderation of the nitronium hexafluorophosphate reactivity by the addition of an amine, such as collidine, may be undertaken for effective conversion to nitrate ester, including trinitroglycerin (NG).

In embodiments of the present disclosure, electrolytic generation of nitronium ion (NO₂⁺) in a divided electrochemical cell from NO₂ and a carrier salt in the presence of an alcohol to provide a nitrate ester may be advantageously undertaken.

For purposes of the present disclosure, various electrode materials may be employed, e.g., glassy carbon or vitreous carbon electrodes may be used that include, e.g., non-graphitizing or non-graphitizable carbon. The noted electrode materials combine glassy and ceramic properties with those of graphite.

In exemplary implementations, isoamyl alcohol (C₅H₁₂O) has been evaluated as a primary alcohol. In initial tests, no nitrate ester products were formed during electrolysis, but nitrite esters were formed quickly due to non-electrochemical reaction between the alcohol and NO₂. When the direct electrolysis of isoamyl alcohol, isoamyl nitrite or 2-propanol was performed with air bubbling, instead of argon, through the reaction, both nitrite ester and nitrate ester were observed in the NMR spectra. Based on the importance of oxygen to the reaction, a radical intermediate may be operative. When direct electrolysis was performed with glycerin using similar methods, a small amount (˜2%) of nitroglycerin was detectible by qNMR, as shown in Table I.

TABLE I
Direct Electrolytic O-Nitration of Alcohol Substrates using
NO₂ with Air Bubbling through the Reaction Solution.

| | | | Consumption | |
|---|---|---|---|---|
| | | | of starting | Yield of |
| | Temp. | Starting | material (% | nitrate ester |
| Reaction | (° C.) | material | conversion) | in situ (%) |
| 1 | 0 | Isoamyl alcohol | 100 * | NA ** |
| 2 | 0 | Isoamyl nitrite | 100 | ~65 |
| 3 | 0 | 2-Propanol | 100 | ~37 |
| 4 | 0 | Glycerol | 100 | ~2 |

* Conversion of isoamyl alcohol to isoamyl nitrite was 100% in the presence of NO₂ giving a clean NMR with no apparent side products; subsequent electrolysis led to complete conversion of isoamyl nitrite to nitrate.
** Yield was not quantified.

Of note, the yield of nitrate ester from the primary alcohol is better than from the secondary alcohol. Also of note, the triol glycerin generated very low yields in the reported tests. Yields were determined by qNMR.

The use of a two-step methodology, where active nitrating agent is first formed electrochemically at the anode followed by use of the anolyte solution in a second reaction step, may be used to form desired nitrated products according to the present disclosure. In exemplary two-step reactions according to the present disclosure, lithium nitrate may be used as a carrier salt to produce N₂O₅ in acetonitrile or other aprotic solvent or solvent mixture, in an anode compartment. The N₂O₅ containing anolyte may be used to simultaneously nitrate both glycerin, to make nitroglycerin (NG), and a secondary alcohol 2-propanol, to make 2-propyl nitrate. The foregoing syntheses may be undertaken without the need for a reactivity modifier, such as Olah's collidine.

Thus, both substrates (i.e., glycerin and 2-propanol) formed nitrate esters, with NG being formed at quantitative in situ yield levels (qNMR) as shown in Table II. The relevant resonances of the NMR spectrum of the NG and 2-propyl nitrate formed in this two-step procedure are shown in FIG. 4. The NMR spectrum shows a clean baseline. The clean baseline indicates no starting glycerol, 2-propanol, or nitrite esters present. The test results demonstrate that 2-propanol was successfully nitrated concurrently with the NG.

TABLE 2
Experimental results where lithium nitrate was used
in conjunction with NO₂ to make N₂O₅ in acetonitrile

| Temp. | Starting | Yield of 1,2,3-Trinitroglycerin |
|---|---|---|
| (° C.) | material | in situ (%) |
| −15 to 20 | Glycerin | 100.0 |

The high NG in situ yield as shown in Table 2 is significant, and may be attributed to N₂O₅ being a milder reagent than nitronium hexafluorophosphate employed by Olah that required modulation with collidine. [See Olah Publication] By way of further example, it has been found that 1,2,4-butanetriol can be converted to 1,2,4-butanetrioltrinitrate (BTTN), an energetic of DOD interest, also in quantitative yield, either in batch nitration or in a flow nitration using an AFR plate.

Alternative alcohol substrates are contemplated for use according to the present disclosure, e.g., ethylene glycol, glycerol, 1,2,4-butanetriol, pentaerythritol, isoamyl alcohol, 2-propanol, glycerol, 2-ethylhexyl alcohol, benzyl alcohol, isosorbide, cyclohexanol, and heterocyclic alcohols such as [3,3′-bis(1,2,4-oxadiaxole)]-5,5′-diyldimethanol. Of note, 2-ethylhexyl alcohol could function as a beneficial fuel additive/cetane improver. In further exemplary embodiments, the alcohol substrate may take the form of an alkyl alcohol, a polyol, a phenol, an anisole and combinations thereof.

O-nitration without the use of N₂O₄ has also been demonstrated according to the present disclosure by direct electrolysis in the anode. As referenced above, the conversion of isoamyl alcohol to its corresponding nitrite ester can be achieved using N₂O₄ without applying electricity. Isoamyl nitrite to nitrate conversion can be achieved electrolytically using AgNO₂ or AgNO₃ on various electrodes (such as Pt, carbon paper, glassy carbon). Experimental reactions were completed using a divided cell, glassy carbon counter electrode, Ag/AgNO₃ (10 mM AgNO₃) reference electrode, 25 mM substrate, 50 mM Ag salts (unless otherwise specified), and 0.1 M TBAPF₆ in ACN.

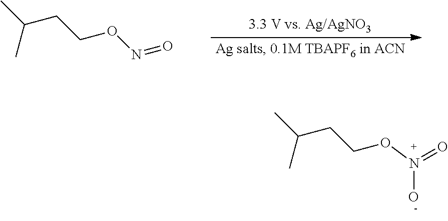

Electrolytic isoamyl nitrite to nitrate conversion demonstrated high yields (72-98%) (see Table 3, entries 1-4) at room temperature and ambient atmosphere. When the electrolysis was conducted using AgOAc as silver salt instead, 52% nitrate ester was obtained (Table 3, entry 5). The observed decrease in product yield may be due to poor solubility of AgOAc in acetonitrile. Several control experiments were conducted to probe the role of Ag ions in the reactions. First, other metal salt with oxidizing power (CuCl₂) was used to replace Ag salts (Table 3, entry 6). Second, the reaction was carried out using AgNO₂ without electricity (Table 3, entry 7). Neither of these two control experiments gave the desired products. The reaction only proceeded in the presence of Ag ions and an electric current. Additionally, only a catalytic amount of AgNO₂ was needed to achieve high product yield, with the aid of electrochemistry (Table 3, entry 8).

TABLE 3
Isoamyl nitrite to isoamyl nitration conversion using Ag salts.

| | | | Yield of isoamyl |
|---|---|---|---|
| | | | nitrate ester |
| Reaction | Electrode | Ag salt | in situ (%)^{a} |
| 1 | Pt | AgNO₂ | 72 |
| 2 | Carbon Paper | AgNO₂ | 78 |
| 3 | GC | AgNO₂ | 97 |
| 4 | GC | AgNO₃ | 98 |
| 5 | GC | AgOAc | 52 |
| 6 | GC | CuCl₂ | 0 |
| 7 | — | AgNO₂ | 0 |
| 8 | GC | 0.2 equiv. (5 mM | 89 |
| | | AgNO₂) | |

^{a}Based on qNMR using 1,2,4,5-tetrachlorobenzene as internal standard. GC standards for glassy carbon

Direct nitration of isoamyl alcohol has been demonstrated according to the present disclosure. Reactions were completed using a divided cell, Ag/AgNO₃ (10 mM AgNO₃) reference electrode, glassy carbon counter electrode, 25 mM substrate, 50 mM Ag salts (unless otherwise specified), 0.1 M TBAPF₆ in ACN, constant potential electrolysis, 3h (unless otherwise specified); room temperature and ambient atmosphere.

AgNO₂ was used as a nitrating reagent and different electrode materials were tested. Glassy carbon (GC) and Pt electrodes showed similar performance, yielding isoamyl nitrate with around 50% yield (Table 4, entries 1-2). A reaction using a carbon paper electrode produced a mixture of its corresponding nitrite and nitrate esters with 59% and 25% product yield, respectively (Table 4, entry 3). Prolonging the electrolysis reaction converted additional isoamyl nitrite to isoamyl nitrate, yielding 68% of isoamyl nitrate at the end of the electrolysis. Notably using a sub-stoichiometric amount of silver ion in combination with another nitrating reagent, such as TBANO₂ achieved 100% conversion (See Table 4, entry 5) with modest yields of nitrate esters. TBANO₂ resulted in more over-oxidation products, as evident by corresponding aldehyde and carboxylic peaks observed in NMR spectra.

The isoamyl nitrate product yield was further improved to 85% by using 10 mM substrates with 20 mM AgNO₂. It is noted that over oxidation products, such as the corresponding aldehyde and carboxylic acid, were observed (Table 4, entries 1-6). Thus, a reaction atmosphere investigation was conducted. Running the reaction under an argon (Ar) environment yielded a further enhancement in product yield, reaching 92% for the nitrate ester (Table 4, entry 7). This improvement can be ascribed to effective mitigation of undesired oxidation products formation through the use of Ar.

TABLE 4
Isoamyl alcohol to isoamyl nitration conversion using AgNO₂.

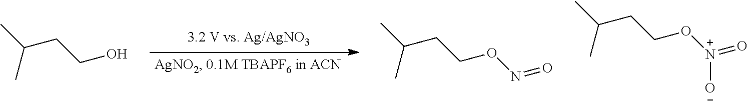

| | | | Substrate | | | Product Yield (%) | |
|---|---|---|---|---|---|---|---|
| | | | conc. | | Conversion | Isoamyl | Isoamyl |
| Entry | Atmosphere | Electrode | (mM) | Nitrating salt | (%) | nitrite | nitrate |
| 1 | Air | Pt | 25 | 50 mM AgNO₂ | 100 | 0 | 51 |
| 2 | Air | Glassy carbon | 25 | 50 mM AgNO₂ | 100 | 0 | 59 |
| | | (GC) | | | | | |
| 3 | Air | Carbon Paper | 25 | 50 mM AgNO₂ | 84 | 59 | 25 |
| 4* | Air | Carbon Paper | 25 | 50 mM AgNO₂ | 100 | 0 | 68 |
| 5 | Air | GC | 10 | 10 mM AgNO₂ | 100 | 0 | 35 |
| | | | | 40 mM | | | |
| | | | | TBANO₂ | | | |
| 6 | Air | GC | 10 | 20 mM AgNO₂ | 100 | 0 | 85 |
| 7 | Ar | GC | 10 | 20 mM AgNO₂ | 100 | 0 | 92 |

Product yield is based on qNMR using 1,2,4,5-tetrachlorobenzene as internal standard;
*Reaction time: 4h.

In addition to isoamyl alcohol, alternative alcohol substrates have been evaluated. Based on such evaluations, it was demonstrated that isopropanol to isopropyl nitrate conversion can be achieved with high yield and selectivity using AgNO₂ as a nitrating reagent in acetonitrile using both Pt and glassy carbon electrodes. Similarly, it was demonstrated that long-chain alcohol 2-ethylhexanol can be effectively converted to its corresponding nitrate and nitrite with 81% and 5% yield, respectively. Of note, 2-ethylhexyl nitrate is used as an important diesel additive to increase the diesel cetane number and to improve combustion performance. Nitration of 1-octanol was also demonstrated, yielding 75% of the corresponding nitrate ester and nitration of isosorbide using AgNO₂ was found to produce a mixture of isosorbide mononitrate and isosorbide dinitrate, with yields of 28% and 11%, respectively. Importantly, isosorbide nitrate esters have been shown to have vasodilatory effects in humans, leading to their application in medical treatments for ailments such as angina. The foregoing results demonstrate that electrochemical nitration of various alcohols to their corresponding nitrate esters with AgNO₂ is a safe and effective modality for generating easy-to-handle nitrogen sources. The nitration of isopropanol and 2-ethylhexanol may be undertaken according to the following reactions (reaction conditions: GC electrode, 10 mM substrate, 20 mM AgNO₂):

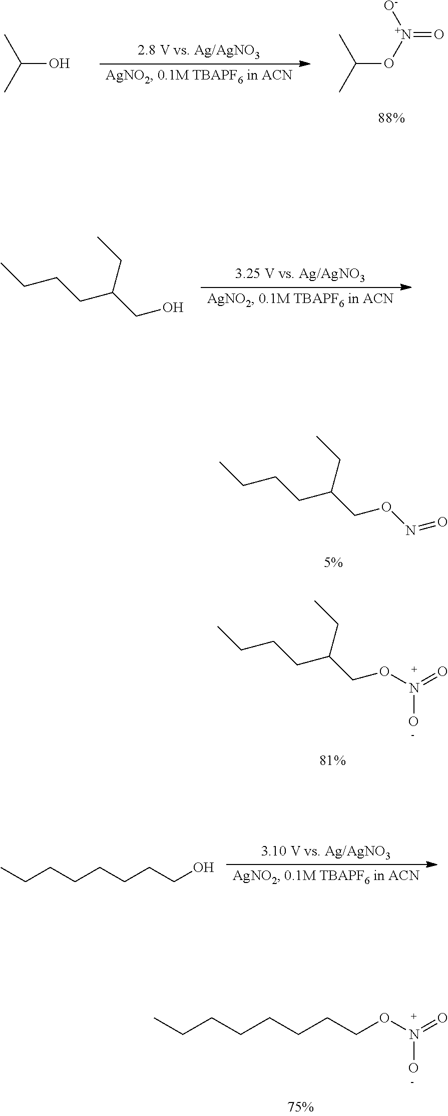

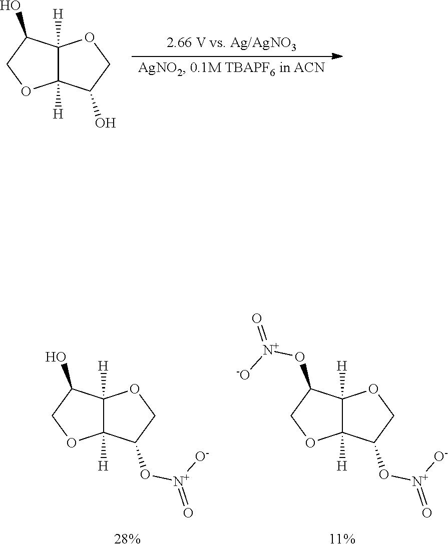

The ability to directly convert nitrite esters to their nitrate esters using silver salts is significant because, inter alia, electrochemically generated N₂O₅ has been shown to be an excellent reagent for conversion of glycerin to 1,2,3-trinitroglycerin, 1,2,4-butanetriol to BTTN and conversion of 2-propanol to 2-propyl nitrate with quantitative in situ yield. Specifically, these results demonstrate that alcohol nitration may be achieved without the use of nitric acid and sulfuric acid. The electrochemical generation of active nitrating species offers a greener alternative to conventional industrial nitration.

The conversion of 1,2,4-butanetriol to BTTN (1,2,4 butanetriol trinitrate) has also been demonstrated. Experimental results described above have been based on divided electrochemical reactions using an H-cell divided with a fine glass frit. Glass frits, which are prone to crack in flow cells, may be replaced with a membrane that allows charged species to pass, but effectively separates cathode from anode.

In further experimental studies according to the present disclosure, a two-piece H-cell was fitted with different membranes using a saturated solution of lithium nitrate in acetonitrile to test membrane conductivity and compared with that of the fine frit H-cell (TABLE). It was found that three of the fluorinated membranes (FP100-300, WP100-100, and WP20-80) performed comparably to the fine glass frit (TABLE).

TABLE 5
Fluorinated membrane dividers used in a two-piece H-cell
test for conductivity using a saturated solution of
lithium nitrate in acetonitrile at room temperature.

| | | Pore size (uM), | Voltage | Current |
|---|---|---|---|---|
| Test | Divider | Thickness (mM) | (V) | (mA) |
| 1 | Fine glass frit | 5, 3000 | 1.8 | 4.0 |
| 2 | WP-20-80 ¹ | 0.2, 80 | 1.9 | 4.4 |
| 3 | FP100-300 ¹ | 1, 300 | 1.7 | 4.5 |
| 4 | WP100-100 ¹ | 1, 100 | 1.7 | 4.3 |
| 5 | LWP-300-75-HOP ^{1, 2} | 30, 75 | 1.9 | 3.7 |
| 6 | HP10-50 ¹ | 0.1, 50 | 0.91 | 0.08 |
| 7 | Vycor (Corning) high- | 0.002, 3810 | 1.7 | 6.4 |
| | silica, high-temperature | | | |
| | glass ³ | | | |

¹ Hydrophobic polytetrafluoroethylene (PTFE) membrane
² Laminated polytetrafluoroethylene (PTFE) membrane
³ Porous glass membrane

In the test results set forth above, three fluorinated membranes demonstrated conductivities comparable to a fine glass frit divided H-cell, as shown in the italicized results (Tests 2-4).

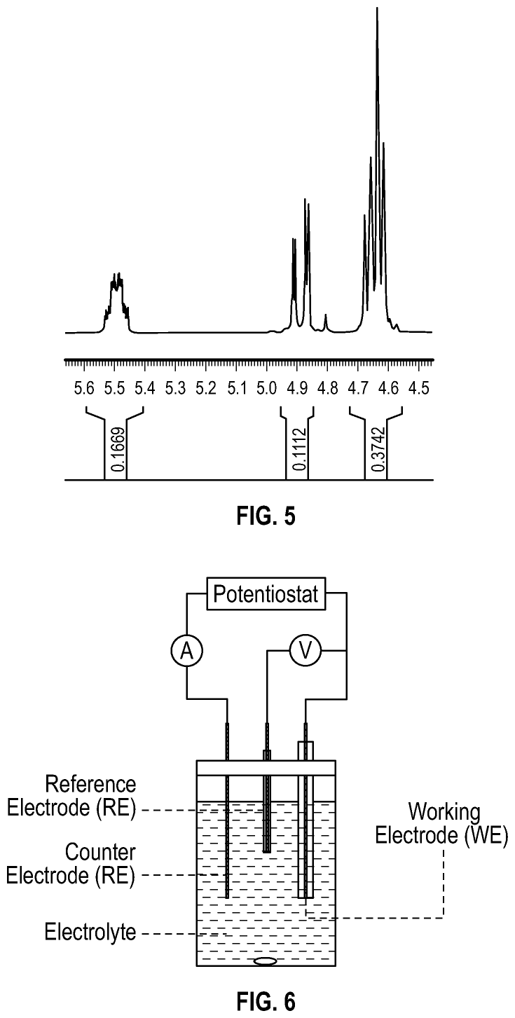

A further experimental electrolysis was undertaken using the FP100-300 membrane as separator with lithium nitrate and N₂O₄ in acetonitrile solvent. The experiment used 0.75 equiv. of nitric acid per half-cell, relative to lithium nitrate, and successfully produced N₂O₅. In the experiment, 2.2 volts and 50 mA of current were delivered, accelerating the formation of N₂O₅ due to the small amount of nitric acid. The anolyte solution was then harvested and added to 1,2,4-butanetriol in acetonitrile at −15° C. to make BTTN in quantitative yield, qNMR, as shown by the NMR spectra set forth in FIG. 5.

These experimental studies demonstrate, inter alia, the efficacy of a continuous flow process according to the present disclosure and further demonstrate that BTTN and NG can be formed effectively using only N₂O₄, a source of electrons, lithium nitrate, and an equivalent of nitric acid to speed the electrolysis, thereby making the reagent N₂O₅ in an aprotic solvent.

When using silver salts (e.g., AgNO₃, AgNO₂), other charge carrying salts including nitrate and nitrite salts (e.g., LiNO₃, TBANO₃, TBANO₂, NaNO₂) may be used to directly nitrate toluene and anisole in acetonitrile (ACN) in the anode compartment according to the present disclosure. TBAPF₆ may be advantageously employed as the supporting electrolyte due to its high electrochemical stability and solubility in ACN. Cyclic voltammetry (CV) may be performed to probe the electrochemical behavior of nitrating reagents and substrates, and to determine suitable potential ranges to be used for bulk electrolysis experiments. In experimental studies, anisole nitration to 2-NA and 4-NA was achieved using AgNO₂ in acetonitrile on various electrode materials (Pt, stainless steel, glassy carbon, carbon paper) in both undivided and divided cells (Table 6).

Experimental conditions were as follows: Pt counter, Ag/AgNO₃ (10 mM AgNO₃) reference electrode, 1.85 V constant potential, 0.1 M TBAPF6 in ACN, 3 h, 50 mM substrate, 50 mM AgNO₂, RT. [The noted method is disclosed in the literature—Electrochimica. Acta. 1975. Vol. 20. Pp. 857-862; however, the anisole nitration disclosed in the literature achieves significantly lesser yield than achieved according to the present disclosure.]

Using the Pt electrode, the reaction in the divided cell (Table 6, entry 2) produced higher product yields (30% 4-NA, 12.5% 2-NA) compared to the reaction in an undivided cell (Table 6, entry 1). Reactions using glassy carbon electrodes showed the same trend (Table 6, entries 5-6). The divided cell setup is effective in preventing unwanted reduction of products on the counter electrode. Electrode material evaluations showed that carbon paper outperformed other electrode materials, yielding 34.1% 4-NA and 19.0% 2-NA (Table 6, entry 7). Of note, carbon paper is a cost-effective and environmentally friendly electrode material.

TABLE 6
Evaluation of different reactor setups and electrode materials

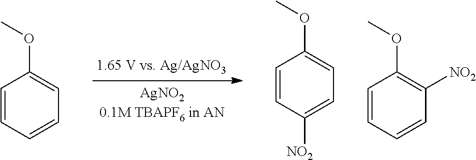

| | | | Product Yield (%)^{a} | |
|---|---|---|---|---|
| Entry | reactor | Electrode materials | 4-NA | 2-NA |
| 1 | Undivided | Pt | 18.8 | 12.5 |
| 2 | Divided | Pt | 30.1 | 17.0 |
| 4 | Undivided | Stainless steel | 14.1 | 10.1 |
| 5 | Undivided | Glassy carbon | 20.4 | 10.5 |
| 6 | Divided | Glassy carbon | 22.8 | 12.1 |
| 7 | Divided | Carbon paper | 34.1 | 19.0 |

^{a}Based on GC analysis with biphenyl as an internal standard

Various reaction conditions have been investigated through experimental study, including the effect of temperature and nitrating salt concentration using a carbon paper electrode (Table 7). Experimental conditions: Divided cell, Carbon paper working electrode, Pt counter, Ag/AgNO₃ (10 mM AgNO₃) reference electrode, 1.85 V constant potential, 0.1 M TBAPF6 in ACN, 3 h, 50 mM substrate. Based on this experimental work, it has been determined that both temperature and AgNO₂ concentration had a positive effect on product yields. The highest product yields (39.1% 4-NA and 25.8% 2-NA) were achieved using 100 mM AgNO₂ at 40° C. (Table 7, entry 5).

TABLE 7
Evaluation of AgNO₂ concentration and
temperature using 50 mM substrate

| | AgNO₂ | | | |
|---|---|---|---|---|
| | concentration | | Product Yield (%)^{a} | |
| Entry | (mM) | Temperature(° C.) | 4-NA | 2-NA |
| 1 | 25 | RT | trace | trace |
| 2 | 50 | RT | 22.8 | 12.1 |
| 3 | 100 | RT | 27.6 | 15.9 |
| 4 | 50 | 40 | 32.6 | 18.0 |
| 5 | 100 | 40 | 39.1 | 25.8 |

^{a}Based on GC analysis with biphenyl as an internal standard

Using AgNO₂ as nitrating reagent, nitration of 4-nitroanisole (4-NA) to its corresponding dinitroanisole (2,4-DNAN) has been demonstrated to achieve selectivity in acetonitrile with various electrode materials (Pt, glassy carbon, and carbon paper) from temperature 0-25° C. (Table 8). Experimental conditions: Divided cell, Pt counter, Ag/AgNO₃ (10 mM AgNO₃) reference electrode, 1.85 V constant potential, 0.1 M TBAHPF in ACN, 3 h, 25 mM substrate, 50 mM AgNO₂. Of note, carbon paper (Table 8, entry 3) was comparable with Pt in terms of product yield (around 50%).

TABLE 8
Evaluation of 4-nitroanisole nitration

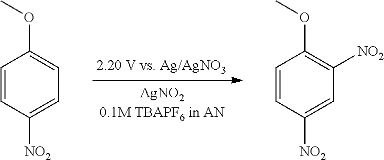

| Entry | Electrode | Temperature (° C.) | 2,4-DNAN yield (%)^{a} |
|---|---|---|---|
| 1 | Pt | RT | 48.8 |
| 2 | Glassy Carbon | RT | 37.3 |
| 3 | Carbon Paper | RT | 49.5 |
| 4 | Carbon Paper | 0 | 50.3 |

^{a}Based on GC analysis with diphenyl as an internal standard

As demonstrated herein, anisole nitration to nitroanisole and dinitroanisole can be achieved using AgNO₂ as a nitrating reagent under mild electrolysis conditions. Although moderate product yield was obtained, the results demonstrate the feasibility/efficacy of nitration of anisole using a safer nitrating reagent, such as AgNO₂. It is noted that Ag plating on the cathode was found. Nevertheless, collecting the Ag plated on the cathode via electrochemical stripping could be a potential solution to address potential cost issues.

As demonstrated herein, Ag-catalyzed electrochemical nitration of alcohol under milder conditions (room temperature and pressure, safe nitrating reagents) offers an effective approach for the production of energetic materials. The disclosed systems and methods advantageously avoid the creation of undesirable “red water waste” (i.e., concentrated HNO₃ and H₂SO₄) as is generated using traditional synthetic routes.

According to the present disclosure, systems and methods are provided wherein a catalytic amount of Ag⁺ and low-cost nitrating reagent (e.g., TBANO₂, TBANO₃, LiNO₃, etc), both primary (isoamyl alcohol) and secondary (isopropanol) alcohol can be converted to the corresponding nitrate with high yield and selectivity. The disclosed systems/methods may be implemented with various alcohols.

In an exemplary reaction setup: a three-electrode system may be employed to provide controlled electrolysis. The electrochemistry may be conducted using a CHI660 Potentiostat, which is capable of recording cyclic voltammograms, as well as conducting electrolysis under either constant current or constant potential conditions with computer-based control and recording of the experiment.

In an exemplary reaction procedure, cyclic voltammetry (CV) may be first carried out using a Potentiostat to probe electrochemical properties of various substrates and various nitrating sources to narrow down the potential windows. Bulk electrolysis may be conducted at specific electrochemical potential (Ep) obtained from CV study. An illustrative implementation of the disclosed reaction procedure is outlined below:
1. Optionally use cyclic voltammetry to determine the electrochemical activity of substrates, e.g., toluene and anisole, and narrow down chemical potential ranges where the substrate is electrochemically active. The working electrode (“WE”) may be, for example, a 3 mm diameter glassy carbon electrode or a 1 mm diameter Pt disk; the counter electrode (“CE”) may be, for example, a 1 mm diameter Pt wire, and the reference electrode (“RE”) may be, for example, Ag/AgNO₃. The CV measurement, which relies on the natural diffusion of analyte, is conducted under rest condition with no stirring.
2. Using information obtained from CV, conduct bulk electrolysis at specific electrochemical potentials. The bulk electrolysis may be performed as follows:
   WE (working electrode): e.g., glassy carbon
   CE (counter electrode): e.g., glassy carbon
   RE (reference electrode): Ag/AgNO₃
   Reaction conditions: stirring applied to maximize mass transfer
3. Alternatively, constant current experiments may be performed when a larger range of potentials is needed to accomplish desired bulk electrolysis testing.

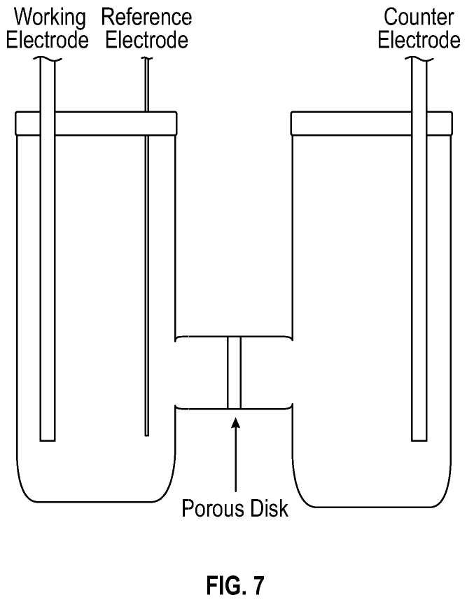

To further illustrate implementation of the disclosed systems/methods, reference is made to the schematic representation of an exemplary reaction setup that includes a divided cell (H-cell) with fine glass frit, as shown in FIG. 7. An advantage of a divided cell as disclosed herein is to separate products (e.g., nitrate esters) from the cathode and thus prevent unwanted side reactions (e.g., reduction of nitrate esters).

According to the present disclosure, reactions may be carried out in an H-cell divided by a fine glass frit (˜5 μM pore size). The anode compartment may be fitted with a working electrode and an Ag/AgNO₃ reference electrode. Both the cathode and anode may be charged with dry ACN. The desired electrolyte (charge-carrying salt) may be added creating a 0.1-0.5 M solution to both cathode and anode chambers. Ag⁺ catalyst (different Ag salts), various nitrating sources (e.g., metal (Ag, Na, K, Li etc), nitrite and nitrate salts, TBANO₂, TBANO₂, etc), and alcohol substrates may be added to the anode chamber. Metal salts (e.g., Ag, Ni, Cu, etc.) may be added to the cathode chamber where metal reduction deposition (e.g., Ag⁺-e⁻→Ag) is the main reaction for the cathode chamber.

### Example of Isoamyl Alcohol Nitration using AgNO₂ as a Nitrating Reagent

The experiments were performed using a two-chambered glass H-cell with fine frit (porosity: 10-20 μm). An oven-dried H-cell divided by a fine glass frit was equipped with stir bars on each side and a glassy carbon with an area of 2 cm² as both anode and cathode material, Ag/AgNO₃ was used as the reference electrode. The distance between the anode and cathode was 4.8 cm.

To both anode and cathode chambers, 20 mL 0.1 M TBAPF₆ in ACN, and 20 mM AgNO₂ were added. 10 mM isoamyl alcohol was subjected to the anode chamber. The electrolysis was conducted using constant potential mode (3.2V vs Ag/AgNO₃) at room temperature and ambient atmosphere for 3 hours. A total 54.4 C of charge was passed during entire electrolysis.

Upon completion of electrolysis, aliquot of the reaction mixture was taken for qNMR analysis with deuterium chloroform as solvent and 1,2,4,5-tetrachlorobenzene as internal standard. A 85% yield of isoamyl nitrate was obtained under air, and a 92% yield of isoamyl nitrate was obtained under Ar. For the reaction under Ar, the solution was degassed with Ar prior to electrolysis and Ar was continuously bubbled during electrolysis.

### Example of Electrolysis in Batch using an H-cell to Produce Active Nitrating Agent which was then used for Nitration of Toluene in a Second Step in Batch

An oven-dried H-cell divided with a fine glass frit was placed under nitrogen and each half cell was equipped with a stir bar. A platinum coil cathode with an area of 0.62 cm², was placed in one half cell compartment and a platinum foil anode with an area of 8.32 cm² was placed in the opposite half cell. The distance between the electrodes was 2.2 cm. The apparatus was cooled to 0° C. in an ice water bath and 23.8 mL of dry acetonitrile was added to each half cell. The anode compartment was fitted with a silver/silver nitrate reference electrode.

Lithium triflate (dried in a vacuum oven over night at 110° C. with N₂ sweep resulting in solids at 0.15 weight % water) was added to the cathode (2.04 g) and anode (2.03 g). Stirring began at 400 RPM and the salts dissolved. After purging with nitrogen for 15 minutes, 24 wt % N₂O₄ in acetonitrile was added to each half cell cathode (3.3 mL) anode (2.7 mL).

Bulk electrolysis using a Pine wavedriver was initiated under constant current conditions at 25 mA, which corresponded to about 2.95 volts versus the reference electrode and about 12.5 volts whole cell. The bulk electrolysis was carried out for 5.25 hours with a total of 482 C of charge or about 5.0 mmol of electrons passing.

A sample of the anolyte was harvested (0.5 mL) and used to nitrate a known excess amount of 4-nitrotoluene to 2,4-dinitrotoluene to measure the amount of nitronium triflate formed by qNMR integration of the respective starting material and product peaks versus an internal standard. The bulk of the anolyte (23 mL) was harvested from the anode and added via syringe pump at 0.5 mL/min to a −15° C. of toluene (77 uL) in acetonitrile (5.0 mL). Once the addition was complete the reaction was allowed to warm to room temperature for over 30 minutes. The solvent was evaporated and the residue was dissolved in methyl tert-butyl ether (MTBE). The organic layer was washed with water and then carbonate solution. The organic layer was evaporated to give dinitrotoluene 112 mg (84% yield) in the following isomer ratios as determined by GC against known standards. 2,6-Dinitrotoluene 35.8%, 2,5-Dinitrotoluene 0.3%, 2,4-Dinitrotoluene 62.0%, 2,3-Dinitrotoluene 1.39%, 3,4-Dinitrotoluene 0.6%.

### Example of Electrolysis in Flow using Micro Flow Cell

Two 100 mL EasyMax reactors with 0.5 M lithium nitrate, 0.5 M N₂O₄, and 0.7 M of nitric acid in acetonitrile at 20° C. An Electrocell Microflow cell (Electrocell North America, Inc., Amherst, NY) was set up with platinum clad niobium electrodes with each electrode having an area of 10 cm² and 0.7 mm apart. Electrolysis was initiated in constant current mode at 900 mA for 39 minutes, then 600 mA for 17 minutes, then constant voltage of 2 V with respect to reference electrode for 2 hours, then constant current of 275 mA for 2 hours. The anolyte produced from this electrolysis was mixed with 2-propanol to produce isopropyl nitrate which when analyzed with qNMR found the concentration of N₂O₅ in the anolyte to be 8 wt %. This solution was then used to nitrate a substrate of anisole.

### Example of Electrolysis in Batch using H-Cell, for Nitration 1,2,4-Butantriol in Flow in a Second Step

An oven dried H-cell divided by a fine glass frit was equipped with stir bars on each side and a platinum coil cathode with an area of 0.62 cm², and platinum foil anode with an area of 8.32 cm². The distance between the electrodes was 2.2 cm. The cells were kept under a nitrogen blanket to keep them dry. The cathode was charged with 15.56 g of 60% ethylene carbonate 40% acetonitrile solution by weight, 5.3 g of 25% NO₂, 45% ethylene carbonate, 30% acetonitrile solution by weight, 0.5 mL of 98% nitric acid and 1.06 g of LiNO₃. The anode was charged with 16.67 g of 60% ethylene carbonate 40% acetonitrile solution by weight, 5.62 g of 25% NO₂ (30.5 mmol), 45% ethylene carbonate, 30% acetonitrile solution by weight, 0.5 mL of 98% nitric acid (12 mmol), and 1.06 g of LiNO₃. A glycol water bath was used to cool the contents of the H-cell to 0° C.

Constant current electrolysis was conducted at 18 mA for 22 hours. In the electrolysis, 70% of the NO₂ was converted to NO₂⁺ producing a 4.9 wt % N₂O₅ solution with a yield of 57%.

For the second step, the nitrating agent (anolyte from electrolysis containing N₂O₅) and substrate (125 mg of 1,2,4-butanetriol in 25 mL of acetonitrile) were pumped by a Harvard syringe pump (Harvard Apparatus, Holliston, MA) at 1.35 mL/min for the product's residence time in the mixing plate to be 1 minute. Both streams then went through Corning AFR low flow plates kept at 0° C. to cool the streams before entering a Corning AFR G1 lab reactor plate kept at 0° C.

Samples were collected every minute once material began exiting the mixing plate until the pumps ran out of material, collecting data on each residence time. These samples were then analyzed with NMR. Nitration streams used a molar ratio of 1:10 1,2,4-butanetriol to N₂O₅. The data taken from the nitration products are set forth in Table 9.

TABLE 9
Molar material balance for the flow nitration of 1,2,4-butanetriol.

| | 1,2,4-Butanetriol | 1,2,4-Butanetriol |
|---|---|---|
| | Trinitrite Yield | Trinitrate Yield |
| Entry | (%) | (%) |
| Sample 2 | 11 | 89 |
| Sample 3 | 0 | 100 |

Table 9 shows that at sample 3, the streams had achieved the residence time necessary to obtain the mixing requirements that would convert all the BT to BTTN without producing any nitrite ester. A quantitative yield of 100% BTTN was achieved for the flow nitration at 5.8 residence times in the mixing plate.

### Example of Electrolysis in Batch using H-Cell, for Nitration Anisole in Flow in a Second Step

An oven dried H-cell divided by a fine glass frit was equipped with stir bars on each side and a platinum coil cathode with an area of 0.62 cm², and platinum foil anode with an area of 8.32 cm². The distance between the electrodes was approximately 2.2 cm. The half cells were kept under a nitrogen blanket to keep them dry. The cathode was charged with 18.49 g of 60% ethylene carbonate 40% acetonitrile solution by weight, 6.11 g of 25% NO₂, 45% ethylene carbonate, 30% acetonitrile solution by weight, and 1.99 g of LiNO₃. The anode was charged with 19.3 g of 60% ethylene carbonate 40% acetonitrile solution by weight, 5.4 g of 25% NO₂ (29.3 mmol), 45% ethylene carbonate, 30% acetonitrile solution by weight, 0.5 mL 98% nitric acid (12 mmol) and 1.99 g of LiNO₃. A glycol water bath was used to cool the contents of the H-cell to 0° C.

Constant current electrolysis was then run at 32 mA for 26 hours. In the electrolysis, 80% of the NO₂ was converted to NO₂⁺ producing a 4.5 wt % N₂O₅ solution with a yield of 46%. For the second step, the nitrating agent (anolyte from electrolysis/N₂O₅) and substrate (481 mg of ansiole in 24 mL of acetonitrile) were pumped by a Harvard syringe pump at 1.35 mL/min. Both streams then went through Corning AFR low flow plates kept at 0° C. to cool the streams before entering a corning AFR G1 lab reactor plate kept at −5° C.

Samples were collected every minute once material began exiting the mixing plate until the pumps ran out of material. These samples were then analyzed with NMR. A ratio of 1:2 anisole to N₂O₅ at 1.35 mL/min. The results of the nitration are shown in Table 10.

TABLE 10
Molar material balance for the flow nitration of anisole.

| | 2,6-Dinitroanisole | 2,4-Dinitroansiole |
|---|---|---|
| Reaction | Yield (%) | Yield (%) |
| Sample 2 | 8 | 83 |
| Sample 3 | 10 | 85 |
| Sample 4 | 9 | 88 |

The data in Table 10 demonstrates that, in this experiment, the yield of 2,4-DNAN was at a maximum during the third sample which spent 4.8 residence times in the mixing plate. This gave a quantitative yield of 88% for 2,4-DNAN and 9% for 2,6-DNAN. The isomer ratio from this synthesis was 10:1 for 2,4-DNAN to 2,6-DNAN.

Although the present disclosure has been provided with reference to exemplary implementations of the disclosed systems and methods, the present disclosure is not limited by or to such exemplary implementations. Rather, the present disclosure is subject to various modifications, refinements and extensions without departing from the spirit or scope of the present disclosure, and such modifications, refinements and extensions are encompassed within the present disclosure.

1. A method for production of energetic and non-energetic nitrogen-containing materials, comprising:
a. providing an electrolysis reaction chamber that includes a reference electrode, a cathode and an anode;
b. introducing a nitrogen source material, an aprotic solvent and a charge-carrying salt to define contents within the electrolysis reaction chamber;
c. electrolyzing the contents of the reaction chamber to generate an active nitrating species; and
d. nitrating a substrate with the active nitrating species to generate an energetic material.

2. The method of claim 1, wherein the nitrogen source material is selected from NO₂ and N₂O₄.

3. The method of claim 1, wherein the nitrogen source comprises a nitrate salt or nitrite salt.

4. The method of claim 3, wherein the nitrate salt or nitrite salt is a silver nitrate salt or a silver nitrite salt.

5. The method of claim 1, wherein the reaction chamber defines an anode chamber and a cathode chamber, and wherein the charge-carrying salt is introduced to at least one of the anode chamber and the cathode chamber.

6. The method of claim 1, wherein the nitrating of the substrate with the active nitrating species occurs directly in an anode compartment defined in the reaction chamber.

7. The method of claim 6, wherein the substrate is introduced to the anode compartment before electrolysis of the contents within the electrolysis reaction chamber commences.

8. The method of claim 6, wherein the substrate is introduced to the anode compartment after electrolysis of the contents within the electrolysis reaction chamber commences.

9. The method of claim 1, wherein the active nitrating species is removed from the reaction chamber and is used to nitrate the substrate to generate the energetic material in a second reaction step.

10. The method of claim 1, wherein the anode is positioned at least in part in an anode compartment adapted to provide an oxidizing environment.

11. The method of claim 10, wherein the oxidizing environment is effective for production of the active nitrating species.

12. The method of claim 1, further comprising introducing an electrolyte to the reaction chamber, wherein the electrolyte is selected from the group consisting of Tetrabutylammonium hexafluorophosphate (TBAPF₆) and congeners with alternative tetraalkyl ammonium cations, and tetrabutylammonium tetrafluoroborate, and congeners with alternative tetraalkyl ammonium cations.

13. (canceled)

14. The method of claim 1, wherein the charge-carrying salt comprises at least one of lithium triflimide, lithium triflate and lithium nitrate.

15. (canceled)

16. (canceled)

17. The method of claim 1, wherein the nitrogen source comprises nitric acid.

18. The method of claim 1, wherein the active nitrating species is selected from a nitrogen-containing cation (nitronium) (NO²⁺) in the form of nitronium hexafluorophosphate, a nitrogen-containing cation (nitronium) (NO²⁺) in the form of nitronium tetrafluoroborate, a nitrogen-containing cation (nitronium) (NO²⁺) in the form of nitronium triflimide, a nitrogen-containing cation (nitronium) (NO²⁺) in the form of nitronium triflate, and a nitrogen-containing cation (nitronium) (NO²⁺) in the form of dinitrogen pentoxide, or N₂O₅.

19. (canceled)

20. (canceled)

21. (canceled)

22. (canceled)

23. The method of claim 1, wherein the aprotic solvent is selected from the group consisting of acetonitrile, ethylene carbonate, propylene carbonate, dimethoxyethane, dimethyl sulfone, sulfolane and combinations thereof.

24. The method of claim 1, wherein the reaction chamber is a divided cell that includes a divider formed of at least one of a fine glass frit and a semi-porous non-reactive hydrophobic or hydrophilic polytetrafluoroethylene membrane.

25. (canceled)

26. (canceled)

27. The method of claim 1, wherein the electrolysis is conducted in a flow cell.

28. (canceled)

29. The method of claim 1, wherein the at least one of the anode and the cathode are fabricated from materials selected from the group consisting of carbon-based materials comprising glassy carbon, carbon nanofiber paper, or graphite, and metals comprising copper, gold, nickel, niobium, tantalum, iridium, platinum, stainless steel, or iridium oxide.

30. The method of claim 1, wherein at least one of the anode and the cathode comprises platinum and/or niobium, and wherein the nitrogen source is selected from NO₂ and N₂O₄.

31. The method of claim 1, wherein the active nitrating species is harvested and used to nitrate an alcohol substrate to form nitrate esters in a separate batch or in a separate flow system in a second nitration step.

32. The method of claim 1, wherein the active nitrating species is harvested and used to nitrate an aromatic substrate to form nitrate esters in a separate batch or in a separate flow system in a second nitration step.

33. The method of claim 1, wherein the substrate is an alcohol and is introduced directly into an anode compartment defined by the reaction chamber.

34. The method of claim 1, wherein the substrate is selected from the group consisting of an alkyl alcohol, a polyol, a phenol, an anisole and combinations thereof.

35. The method of claim 1, wherein the substrate is selected from the group consisting of ethylene glycol, glycerol, 1,2,4-butanetriol, pentaerythritol, isoamyl alcohol, 2-propanol, glycerol, 2-ethylhexyl alcohol, benzyl alcohol, isosorbide, cyclohexanol, a heterocyclic alcohol, and [3,3′-bis(1,2,4-oxadiaxole)]-5,5′-diyldimethanol.

36. The method of claim 1, wherein the substrate is an aromatic substrate and is introduced directly into an anode compartment defined by the reaction chamber.

37. The method of claim 1, wherein the active nitrating species is harvested and used to nitrate an aromatic substrate to form nitroaromatic products in a separate batch or in a separate flow system in a second nitration step.

38. The method of claim 1, wherein the substrate is selected from the group consisting of anisole, 4-nitroanisole, toluene, 4-nitrotoluene, 2-nitrotoluene, benzene, chlorobenzene, phenol, pyrazole, furan, thiophene, aniline, N-methylaniline, dimethylaniline, and stilbene.
